(19) United States
(12) Patent Application Publication
(10) Pub. No.: US 2016/0296174 A1
(43) Pub. Date: Oct. 13, 2016

(54) METHOD OF REDUCING MOTION ARTIFACTS ON WEARABLE OPTICAL SENSOR DEVICES

(71) Applicant: APPLE INC., Cupertino, CA (US)

(72) Inventors: Serhan O. ISIKMAN, Sunnyvale, CA (US); Nevzat Akin KESTELLI, San Jose, CA (US); Brian R. LAND, Woodside, CA (US); Albert WANG, Sunnyvale, CA (US)

(21) Appl. No.: 15/100,626

(22) PCT Filed: Dec. 5, 2013
(86) PCT No.: PCT/US2013/073400

§ 371 Date: May 31, 2016

(51) Int. Cl. A61B 5/00; A61B 5/024

(52) U.S. Cl. CPC A61B 5/7214; A61B 5/02416; A61B 5/7278; A61B 5/681; A61B 5/7475; A61B 2562/0233

(57) ABSTRACT

A PPG signal may be obtained from a pulse oximeter, which employs a light emitter and a light sensor to measure the perfusion of blood to the skin of a user. However, the signal may be compromised by noise due to motion artifacts. That is, movement of the body of a user may cause a gap between the tissue of a user and the electronic device, introducing noise to the signal. Further, the noise introduced may vary depending on how close the light emitter is to the light sensor. Accordingly, to address the presence of motion artifacts, examples of the present disclosure can receive light information at a light sensor from two different light emitters, each at a different distance from the light sensor along a surface of the electronic device, one relatively close to the light sensor and one relatively far from the light sensor.

### FIELD OF THE DISCLOSURE

This relates generally to reducing motion artifacts from a photoplethysmogram (PPG) signal.

### BACKGROUND OF THE DISCLOSURE

A PPG signal may be obtained from a pulse oximeter, which employs a light emitter and a light sensor to measure the perfusion of blood to the skin of a user. However, the signal may be compromised by noise due to motion artifacts. That is, movement of the body of a user may cause a gap between the tissue of a user and the electronic device, introducing noise to the signal.

### SUMMARY OF THE DISCLOSURE

A PPG signal may be obtained from a pulse oximeter, which employs a light emitter and a light sensor to measure the perfusion of blood to the skin of a user. However, the signal may be compromised by noise due to motion artifacts. That is, movement of the body of a user may cause a gap between the tissue of a user and the electronic device, introducing noise to the signal. Further, the noise introduced may vary depending on how close the light emitter is to the light sensor. Accordingly, to address the presence of motion artifacts, examples of the present disclosure can receive light information at a light sensor from two different light emitters, each at a different distance from the light sensor along a surface of the electronic device, one relatively close to the light sensor and one relatively far from the light sensor. The sensed light from the relatively close light emitter may decrease as a gap between the electronic device and the tissue increases. In contrast, the sensed light from the relatively far light emitter may increase as the gap increases. A combination (e.g., linear) of the light information corresponding to each light emitter can thereby reduce the presence of noise in the heart rate signal due to changes in the gap between the electronic device and the tissue.

### BRIEF DESCRIPTION OF THE DRAWINGS

FIG. 1A illustrates an electronic device having light emitters and a light sensor for determining a heart rate signal according to examples of the disclosure.

FIG. 1B illustrates an exemplary graph illustrating variation in light signals from first and second emitters as a function of distance from tissue of a user according to examples of the disclosure.

FIG. 2 illustrates a method of computing a heart rate signal according to examples of the disclosure.

FIG. 3 illustrates an electronic device having light emitters and light sensors for determining a heart rate signal according to examples of the disclosure.

FIG. 4 is a block diagram illustrating an exemplary API architecture, which may be used in some examples of the disclosure.

FIG. 5 illustrates an exemplary software stack of an API according to examples of the disclosure.

FIG. 6 is a block diagram illustrating exemplary interactions between the touch screen and other components of the device according to examples of the disclosure.

FIG. 7 is a block diagram illustrating an example of a system architecture that may be embodied within any portable or non-portable device according to examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

A PPG signal may be obtained from a pulse oximeter, which employs a light emitter and a light sensor to measure the perfusion of blood to the skin of a user. However, the signal may be compromised by noise due to motion artifacts. That is, movement of the body of a user may cause a gap between the tissue of a user and the electronic device, introducing noise to the signal. Further, the noise introduced may vary depending on how close the light emitter is to the light sensor. Accordingly, to address the presence of motion artifacts, examples of the present disclosure can receive light information at a light sensor from two different light emitters, each at a different distance from the light sensor along a surface of the electronic device, one relatively close to the light sensor and one relatively far from the light sensor. The sensed light from the relatively close light emitter may decrease as a gap between the electronic device and the tissue increases. In contrast, the sensed light from the relatively far light emitter may increase as the gap increases. A combination (e.g., linear) of the light information corresponding to each light emitter can thereby reduce the presence of noise in the heart rate signal due to changes in the gap between the electronic device and the tissue.

Although examples disclosed herein may be described and illustrated primarily in terms of two emitters and a single sensor, it should be understood that the examples are not so limited, but are additionally applicable to devices including any number of sensors and emitters.

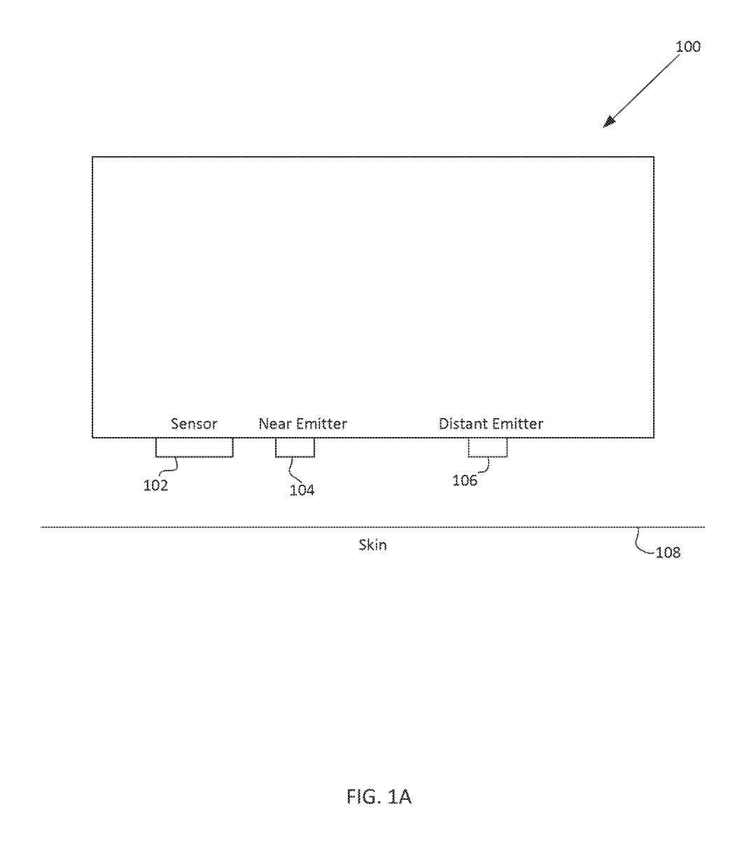

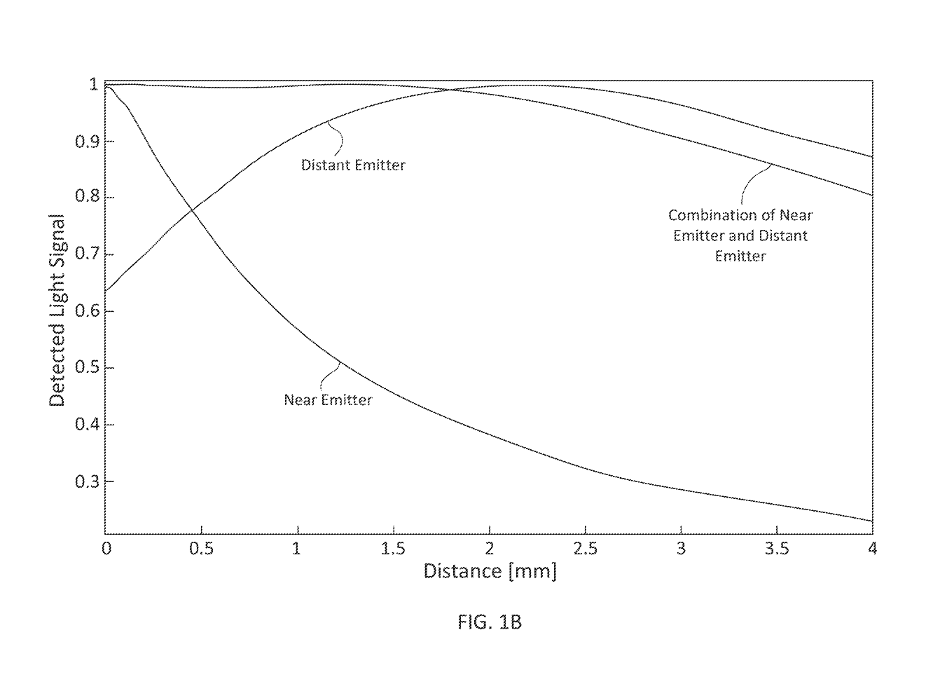

FIG. 1A illustrates an electronic device 100 having a light sensor 102 and light emitters 104 and 106 for determining a heart rate signal according to examples of the disclosure. A light sensor 102 may be co-located with a first light emitter 104 on a surface of the electronic device 100. Additionally, a second light emitter 106 may be located on a surface of the electronic device 100. The distant second light emitter 106 may be further from the light sensor 102 than the near first light emitter 104 along a surface of the electronic device 100. The electronic device 100 may be situated such that the sensor 102 and the emitters 104 and 106 are proximate the skin 108 of a user, so that light from a light emitter can be incident on the skin. For example, the electronic device 100 may be held in a user's hand or strapped to a user's wrist, among other possibilities.

A portion of the light from the light emitters 104 and 106 may be absorbed by the skin, vasculature, and/or blood, among other possibilities, and a portion may be reflected back to the light sensor 102. The amount of light reflected back to the light sensor 102 may vary based on a gap between the electronic device 100 and the skin 108, introducing noise into a heart rate signal computed based on the sensed light information. For the first light emitter 104, the amount of light reflected back to the light sensor 102 may decrease as the gap increases. In contrast, for a light emitter further away from the light sensor 102, such as second light emitter 106, the amount of light reflected back to the light sensor 102 may increase as the gap increases. Accordingly, a heart rate signal computed based on a linear combination of the light information from the first light sensor 104 and light information from the second light sensor 106 may have reduced noise due to variations in the gap between the electronic device 100 and the skin 108.

This effect is further illustrated in FIG. 1B, which shows in an example how gap distance can affect the light signal detected at a light sensor. The figure contrasts the light signal from a Near Emitter that is relatively near the light sensor with the light signal from a Distant Emitter that is relatively distant from the light sensor. Within the first 2 millimeters, the light signal from the Distant Emitter may increase as the gap distance increases, and the light signal may begin to decrease after about 2.5 millimeters. In contrast, the light signal from the Near Emitter may decrease as the gap distance increases. Accordingly, as illustrated in FIG. 1B, a combination of signals from the Near Emitter and the Distant Emitter may be relatively static within the first 2 millimeters of gap distance, and thus this combination can be used to approximate a heart rate signal having reduced noise due to fluctuations in the gap distance.

The precise distances between each emitter and the light sensor may need to be chosen such that the noise can be properly reduced. In some examples, the distance between the light sensor 102 and the first light emitter may be up to 3 mm, and the distance between the light sensor and the second light emitter may be up to 5 mm. Further, an angle of emission of each light emitter may need to be chosen such that noise can be properly reduced. In some examples, the first light emitter 104 may have an emission pattern up to +/−80 degrees, and the second light emitter 106 may have an emission pattern up to +/−70 degrees. It may be beneficial in some examples for the second light emitter to have a more acute angle of emission than that of the first light emitter, the second light emitter being farther from the light sensor than the first light emitter.

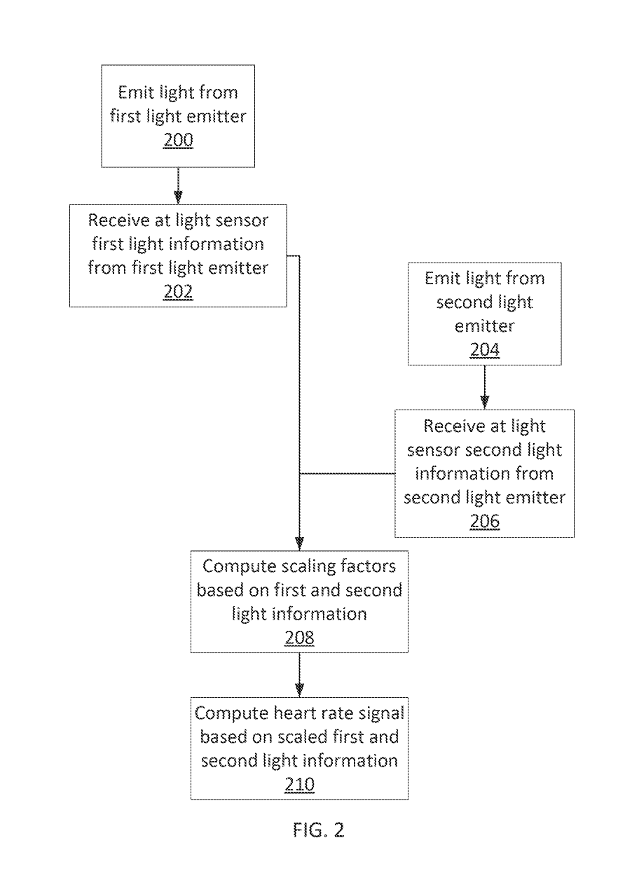

FIG. 2 illustrates a method of computing a heart rate signal according to examples of the disclosure. Light may be emitted from a first light emitter (200). As illustrated in FIG. 1A, first and second light emitters may be on the surface of an electronic device, along with a light sensor. First light information may be received at a light sensor from the first light emitter (202). Light may emitted from a second light emitter (204), and second light information from the second light emitter may be received at the light sensor (206). In some examples, a light emitter may emit light, the light may travel to the skin of a user, and a portion of the light may reflect to a light sensor. Accordingly, the first light information may indicate an amount of light from a first light emitter that has been reflected by the skin, blood, and/or vasculature of the user, among other possibilities. In some examples, the first light information may indicate an amount of light from the first light emitter that has been absorbed by the skin, blood, and/or vasculature of the user.

The light emitters may produce light in ranges corresponding to infrared (IR), green, amber, blue, and/or red light, among other possibilities. Additionally, the light sensors may be configured to sense light having certain wavelengths more easily than light having other wavelengths (e.g., the light sensors can be tuned to those wavelengths). For example, if the first light emitter emits light having a wavelength in the IR range, then the first light sensor may be configured to sense light in the IR range more easily than light in the green range. That is, the incidence of light in the IR range may produce a stronger response in the first light sensor than the incidence of light in the green range. In this way, the first light sensor can be configured so as to sense the light produced by the first light emitter more easily than the light produced by the second light emitter, for example. In some examples, each light emitter may produce light in the same wavelength.

In some examples, a light emitter may be a light emitting diode (LED) and a light sensor may be a photodiode. The light information may include information produced by the photodiode. For example, the light information may include a voltage reading that corresponds to light absorbed by the photodiode. In some examples, the light information may include some transformation of raw signal produced by the photodiode, such as through filtering, scaling, or other signal processing.

In some examples, the emission of light from the first and second light emitters may be time-divided such that the emission of light is alternated between the two light emitters. Thereby, the light information received at the light sensor during a first period can be associated with the first light emitter, and the light information received at the light sensor during a second period can be associated with the second light emitter.

Scaling factors may be computed based on the first and second light information (208). Before the first light information is combined (e.g., added, subtracted, etc.) with the second light information to compute the heart rate signal, the first and second light information may each need to be scaled by a scaling factor. The scaling factors may be computed based on the first and second light information and continuously optimized in real time using smart optimization algorithms to reduce motion artifacts. That is, the scaling factors may be optimized based on previous first and second light information and a previously computed heart rate signal. For example, because noise from variations in gap distance may be orders of magnitude greater than a heart rate signal, the space of possible scaling factors may be searched using an optimization algorithm such that the magnitude of the signal itself is minimized—by minimizing the signal, the noise may be effectively minimized.

Based on the scaled first and second light information, a heart rate signal may be computed (210). For example, each light information may be scaled by a corresponding scaling factor and the scaled first light information may be added to the scaled second light information to obtain a heart rate signal.

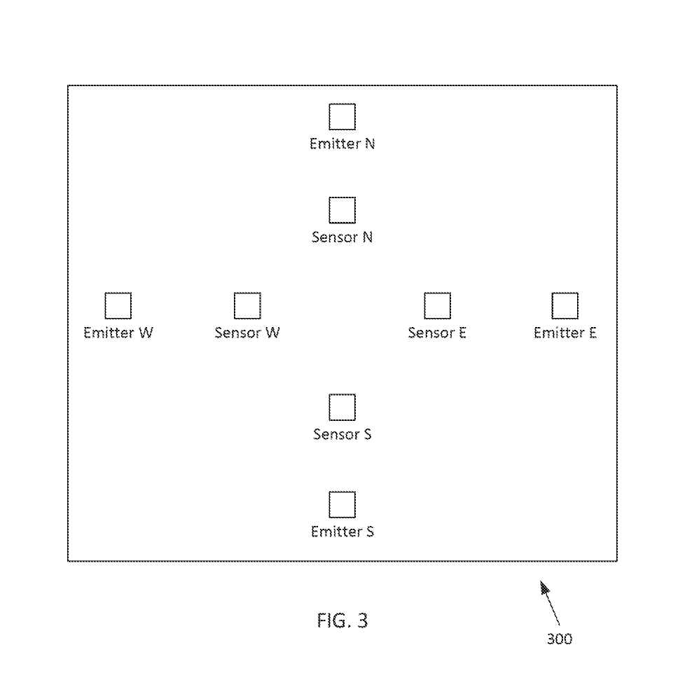

FIG. 3 illustrates an exemplary configuration of a plurality of sensors and a plurality of emitters for computing a heart rate signal having reduced noise due to gap distance variation. Each of the sensors and emitters may be arranged on a surface 300 of an electronic device. Emitter W, Sensor W, Sensor E, and Emitter E may be arranged in a line along the surface 300, and the method illustrated in FIG. 2 may be performed once with Emitter W as a near emitter and Emitter E as a distant emitter with respect to Sensor W, and then again with Emitter E as a near emitter and Emitter W as a distant emitter with respect to Sensor E. The four signals may be combined (e.g., linear combination) and four scaling factors may be chosen using an optimization algorithm to compute a heart rate signal. Similarly, Emitter N, Sensor N, Sensor S, and Emitter S may be arranged in a line along the surface 300, and the method illustrated in FIG. 2 may be performed once with Emitter N as a near emitter and Emitter S as a distant emitter with respect to Sensor N, and then again with Emitter S as a near emitter and Emitter N as a distant emitter with respect to Sensor S. The four signals may be combined with the four signals from Sensors W and E, and eight scaling factors may be chosen using an optimization algorithm to compute a heart rate signal. In this way, any number of sensors and emitters may be used to implement the method of FIG. 2. Furthermore, FIG. 3 illustrates a way to use a single emitter as both a near and distant emitter for different sensors (e.g., in the example above, Emitter E may be used as a near emitter for Sensor E and a distant emitter for Sensor W). This can further save time and power because each time an emitter emits light, the light can be sensed by multiple sensors (e.g., in the example above, the light from Emitter E may be sensed by both Sensor E and Sensor W).

The examples discussed above can be implemented in one or more Application Programming Interfaces (APIs). An API is an interface implemented by a program code component or hardware component (hereinafter “API-implementing component”) that allows a different program code component or hardware component (hereinafter “API-calling component”) to access and use one or more functions, methods, procedures, data structures, classes, and/or other services provided by the API-implementing component. An API can define one or more parameters that are passed between the API-calling component and the API-implementing component.

The above-described features can be implemented as part of an application program interface (API) that can allow it to be incorporated into different applications (e.g., spreadsheet apps) utilizing touch input as an input mechanism. An API can allow a developer of an API-calling component (which may be a third party developer) to leverage specified features, such as those described above, provided by an API-implementing component. There may be one API-calling component or there may be more than one such component. An API can be a source code interface that a computer system or program library provides in order to support requests for services from an application. An operating system (OS) can have multiple APIs to allow applications running on the OS to call one or more of those APIs, and a service (such as a program library) can have multiple APIs to allow an application that uses the service to call one or more of those APIs. An API can be specified in terms of a programming language that can be interpreted or compiled when an application is built.

In some examples, the API-implementing component may provide more than one API, each providing a different view of the functionality implemented by the API-implementing component, or with different aspects that access different aspects of the functionality implemented by the API-implementing component. For example, one API of an API-implementing component can provide a first set of functions and can be exposed to third party developers, and another API of the API-implementing component can be hidden (not exposed) and provide a subset of the first set of functions and also provide another set of functions, such as testing or debugging functions which are not in the first set of functions. In other examples the API-implementing component may itself call one or more other components via an underlying API and thus be both an API-calling component and an API-implementing component.

An API defines the language and parameters that API-calling components use when accessing and using specified features of the API-implementing component. For example, an API-calling component accesses the specified features of the API-implementing component through one or more API calls or invocations (embodied for example by function or method calls) exposed by the API and passes data and control information using parameters via the API calls or invocations. The API-implementing component may return a value through the API in response to an API call from an API-calling component. While the API defines the syntax and result of an API call (e.g., how to invoke the API call and what the API call does), the API may not reveal how the API call accomplishes the function specified by the API call. Various API calls are transferred via the one or more application programming interfaces between the calling (API-calling component) and an API-implementing component. Transferring the API calls may include issuing, initiating, invoking, calling, receiving, returning, or responding to the function calls or messages; in other words, transferring can describe actions by either of the API-calling component or the API-implementing component. The function calls or other invocations of the API may send or receive one or more parameters through a parameter list or other structure. A parameter can be a constant, key, data structure, object, object class, variable, data type, pointer, array, list or a pointer to a function or method or another way to reference a data or other item to be passed via the API.

Furthermore, data types or classes may be provided by the API and implemented by the API-implementing component. Thus, the API-calling component may declare variables, use pointers to, use or instantiate constant values of such types or classes by using definitions provided in the API.

Generally, an API can be used to access a service or data provided by the API-implementing component or to initiate performance of an operation or computation provided by the API-implementing component. By way of example, the API-implementing component and the API-calling component may each be any one of an operating system, a library, a device driver, an API, an application program, or other module (it should be understood that the API-implementing component and the API-calling component may be the same or different type of module from each other). API-implementing components may in some cases be embodied at least in part in firmware, microcode, or other hardware logic. In some examples, an API may allow a client program to use the services provided by a Software Development Kit (SDK) library. In other examples an application or other client program may use an API provided by an Application Framework. In these examples the application or client program may incorporate calls to functions or methods provided by the SDK and provided by the API or use data types or objects defined in the SDK and provided by the API. An Application Framework may in these examples provide a main event loop for a program that responds to various events defined by the Framework. The API allows the application to specify the events and the responses to the events using the Application Framework. In some implementations, an API call can report to an application the capabilities or state of a hardware device, including those related to aspects such as input capabilities and state, output capabilities and state, processing capability, power state, storage capacity and state, communications capability, etc., and the API may be implemented in part by firmware, microcode, or other low level logic that executes in part on the hardware component.

The API-calling component may be a local component (i.e., on the same data processing system as the API-implementing component) or a remote component (i.e., on a different data processing system from the API-implementing component) that communicates with the API-implementing component through the API over a network. It should be understood that an API-implementing component may also act as an API-calling component (i.e., it may make API calls to an API exposed by a different API-implementing component) and an API-calling component may also act as an API-implementing component by implementing an API that is exposed to a different API-calling component.

The API may allow multiple API-calling components written in different programming languages to communicate with the API-implementing component (thus the API may include features for translating calls and returns between the API-implementing component and the API-calling component); however the API may be implemented in terms of a specific programming language. An API-calling component can, in one example, call APIs from different providers such as a set of APIs from an OS provider and another set of APIs from a plug-in provider and another set of APIs from another provider (e.g. the provider of a software library) or creator of the another set of APIs.

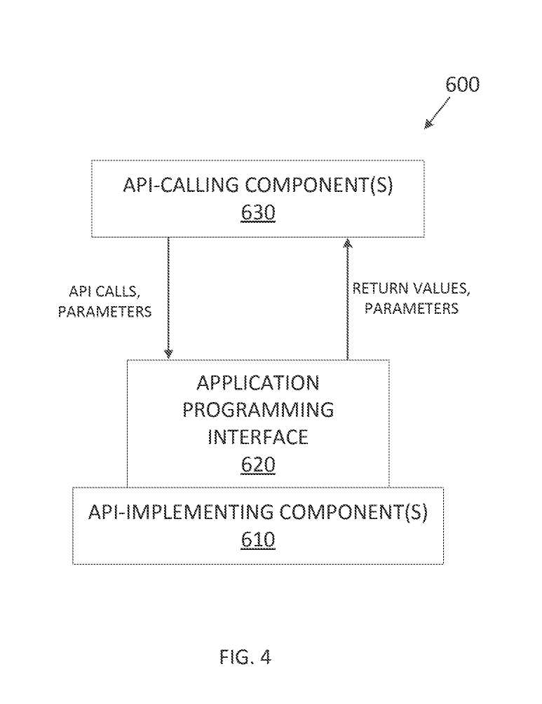

FIG. 4 is a block diagram illustrating an exemplary API architecture, which may be used in some examples of the disclosure. As shown in FIG. 4, the API architecture 600 includes the API-implementing component 610 (e.g., an operating system, a library, a device driver, an API, an application program, software or other module) that implements the API 620. The API 620 specifies one or more functions, methods, classes, objects, protocols, data structures, formats and/or other features of the API-implementing component that may be used by the API-calling component 630. The API 620 can specify at least one calling convention that specifies how a function in the API-implementing component receives parameters from the API-calling component and how the function returns a result to the API-calling component. The API-calling component 630 (e.g., an operating system, a library, a device driver, an API, an application program, software or other module), makes API calls through the API 620 to access and use the features of the API-implementing component 610 that are specified by the API 620. The API-implementing component 610 may return a value through the API 620 to the API-calling component 630 in response to an API call.

It will be appreciated that the API-implementing component 610 may include additional functions, methods, classes, data structures, and/or other features that are not specified through the API 620 and are not available to the API-calling component 630. It should be understood that the API-calling component 630 may be on the same system as the API-implementing component 610 or may be located remotely and accesses the API-implementing component 610 using the API 620 over a network. While FIG. 4 illustrates a single API-calling component 630 interacting with the API 620, it should be understood that other API-calling components, which may be written in different languages (or the same language) than the API-calling component 630, may use the API 620.

The API-implementing component 610, the API 620, and the API-calling component 630 may be stored in a non-transitory machine-readable storage medium, which includes any mechanism for storing information in a form readable by a machine (e.g., a computer or other data processing system). For example, a machine-readable medium includes magnetic disks, optical disks, random access memory; read only memory, flash memory devices, etc.

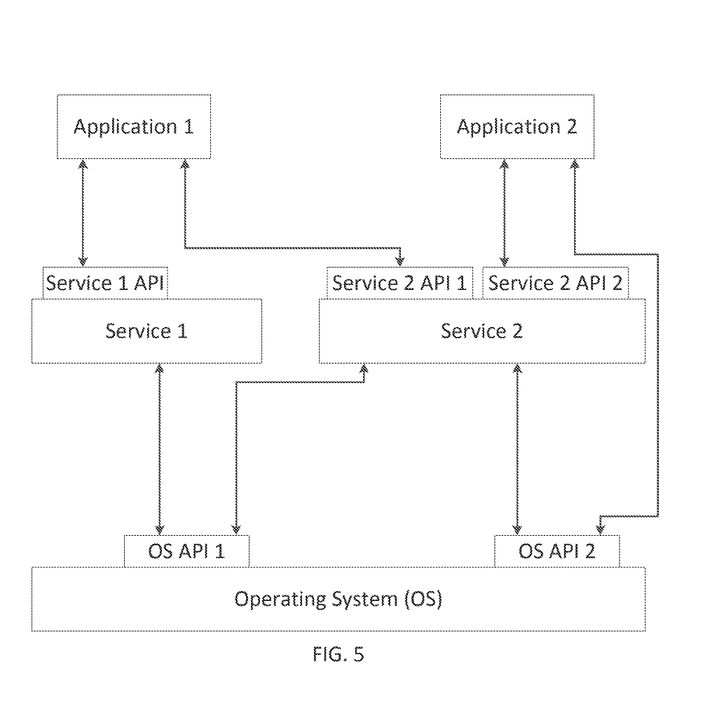

In the exemplary software stack shown in FIG. 5, applications can make calls to Services A or B using several Service APIs and to Operating System (OS) using several OS APIs. Services A and B can make calls to OS using several OS APIs.

Note that the Service 2 has two APIs, one of which (Service 2 API 1) receives calls from and returns values to Application 1 and the other (Service 2 API 2) receives calls from and returns values to Application 2. Service 1 (which can be, for example, a software library) makes calls to and receives returned values from OS API 1, and Service 2 (which can be, for example, a software library) makes calls to and receives returned values from both OS API 1 and OS API 2. Application 2 makes calls to and receives returned values from OS API 2.

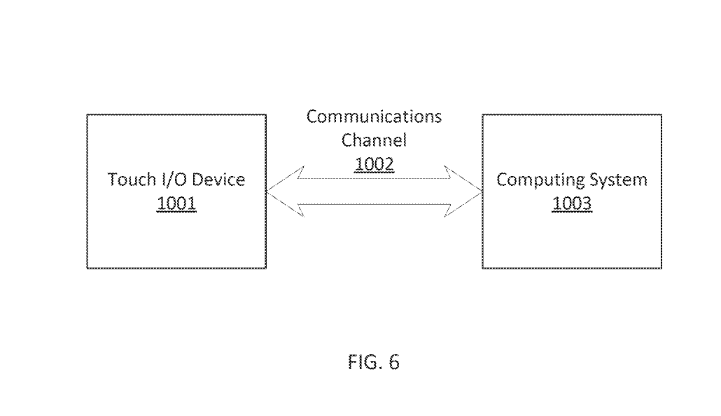

FIG. 6 is a block diagram illustrating exemplary interactions between the touch screen and the other components of the device. Described examples may include touch I/O device 1001 that can receive touch input for interacting with computing system 1003 via wired or wireless communication channel 1002. Touch I/O device 1001 may be used to provide user input to computing system 1003 in lieu of or in combination with other input devices such as a keyboard, mouse, etc. One or more touch I/O devices 1001 may be used for providing user input to computing system 1003. Touch I/O device 1001 may be an integral part of computing system 1003 (e.g., touch screen on a smartphone or a tablet PC) or may be separate from computing system 1003.

Touch I/O device 1001 may include a touch sensing panel which is wholly or partially transparent, semitransparent, non-transparent, opaque or any combination thereof. Touch I/O device 1001 may be embodied as a touch screen, touch pad, a touch screen functioning as a touch pad (e.g., a touch screen replacing the touchpad of a laptop), a touch screen or touchpad combined or incorporated with any other input device (e.g., a touch screen or touchpad disposed on a keyboard) or any multi-dimensional object having a touch sensing surface for receiving touch input.

In one example, touch I/O device 1001 embodied as a touch screen may include a transparent and/or semitransparent touch sensing panel partially or wholly positioned over at least a portion of a display. According to this example, touch I/O device 1001 functions to display graphical data transmitted from computing system 1003 (and/or another source) and also functions to receive user input. In other examples, touch I/O device 1001 may be embodied as an integrated touch screen where touch sensing components/devices are integral with display components/devices. In still other examples a touch screen may be used as a supplemental or additional display screen for displaying supplemental or the same graphical data as a primary display and to receive touch input.

Touch I/O device 1001 may be configured to detect the location of one or more touches or near touches on device 1001 based on capacitive, resistive, optical, acoustic, inductive, mechanical, chemical measurements, or any phenomena that can be measured with respect to the occurrences of the one or more touches or near touches in proximity to device 1001. Software, hardware, firmware or any combination thereof may be used to process the measurements of the detected touches to identify and track one or more gestures. A gesture may correspond to stationary or non-stationary, single or multiple, touches or near touches on touch I/O device 1001. A gesture may be performed by moving one or more fingers or other objects in a particular manner on touch I/O device 1001 such as tapping, pressing, rocking, scrubbing, twisting, changing orientation, pressing with varying pressure and the like at essentially the same time, contiguously, or consecutively. A gesture may be characterized by, but is not limited to a pinching, sliding, swiping, rotating, flexing, dragging, or tapping motion between or with any other finger or fingers. A single gesture may be performed with one or more hands, by one or more users, or any combination thereof.

Computing system 1003 may drive a display with graphical data to display a graphical user interface (GUI). The GUI may be configured to receive touch input via touch I/O device 1001. Embodied as a touch screen, touch I/O device 1001 may display the GUI. Alternatively, the GUI may be displayed on a display separate from touch I/O device 1001. The GUI may include graphical elements displayed at particular locations within the interface. Graphical elements may include but are not limited to a variety of displayed virtual input devices including virtual scroll wheels, a virtual keyboard, virtual knobs, virtual buttons, any virtual UI, and the like. A user may perform gestures at one or more particular locations on touch I/O device 1001 which may be associated with the graphical elements of the GUI. In other examples, the user may perform gestures at one or more locations that are independent of the locations of graphical elements of the GUI. Gestures performed on touch I/O device 1001 may directly or indirectly manipulate, control, modify, move, actuate, initiate or generally affect graphical elements such as cursors, icons, media files, lists, text, all or portions of images, or the like within the GUI. For instance, in the case of a touch screen, a user may directly interact with a graphical element by performing a gesture over the graphical element on the touch screen. Alternatively, a touch pad generally provides indirect interaction. Gestures may also affect non-displayed GUI elements (e.g., causing user interfaces to appear) or may affect other actions within computing system 1003 (e.g., affect a state or mode of a GUI, application, or operating system). Gestures may or may not be performed on touch I/O device 1001 in conjunction with a displayed cursor. For instance, in the case in which gestures are performed on a touchpad, a cursor (or pointer) may be displayed on a display screen or touch screen and the cursor may be controlled via touch input on the touchpad to interact with graphical objects on the display screen. In other examples in which gestures are performed directly on a touch screen, a user may interact directly with objects on the touch screen, with or without a cursor or pointer being displayed on the touch screen.

Feedback may be provided to the user via communication channel 1002 in response to or based on the touch or near touches on touch I/O device 1001. Feedback may be transmitted optically, mechanically, electrically, olfactory, acoustically, or the like or any combination thereof and in a variable or non-variable manner.

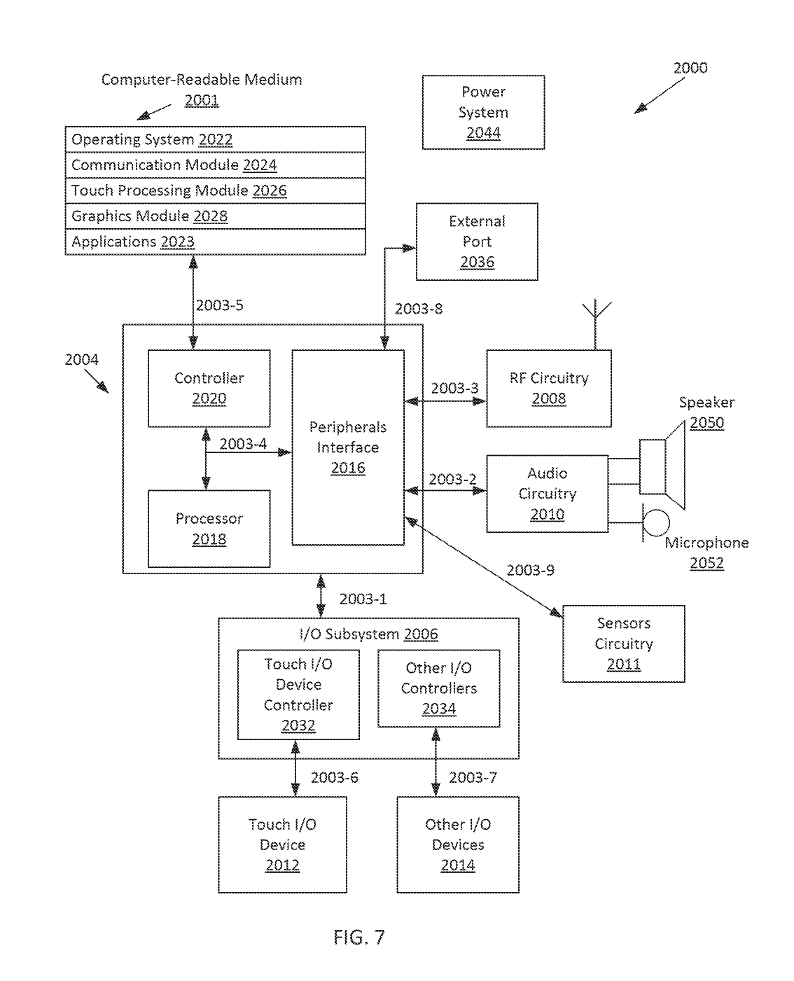

Attention is now directed towards examples of a system architecture that may be embodied within any portable or non-portable device including but not limited to a communication device (e.g. mobile phone, smart phone), a multi-media device (e.g., MP3 player, TV, radio), a portable or handheld computer (e.g., tablet, netbook, laptop), a desktop computer, an All-In-One desktop, a peripheral device, or any other system or device adaptable to the inclusion of system architecture 2000, including combinations of two or more of these types of devices. FIG. 7 is a block diagram of one example of system 2000 that generally includes one or more computer-readable mediums 2001, processing system 2004, I/O subsystem 2006, radio frequency (RF) circuitry 2008, audio circuitry 2010, and sensors circuitry 2011. These components may be coupled by one or more communication buses or signal lines 2003.

It should be apparent that the architecture shown in FIG. 7 is only one example architecture of system 2000, and that system 2000 could have more or fewer components than shown, or a different configuration of components. The various components shown in FIG. 7 can be implemented in hardware, software, firmware or any combination thereof, including one or more signal processing and/or application specific integrated circuits.

RF circuitry 2008 can be used to send and receive information over a wireless link or network to one or more other devices and includes well-known circuitry for performing this function. RF circuitry 2008 and audio circuitry 2010 can be coupled to processing system 2004 via peripherals interface 2016. Interface 2016 can include various known components for establishing and maintaining communication between peripherals and processing system 2004. Audio circuitry 2010 can be coupled to audio speaker 2050 and microphone 2052 and can include known circuitry for processing voice signals received from interface 2016 to enable a user to communicate in real-time with other users. In some examples, audio circuitry 2010 can include a headphone jack (not shown). Sensors circuitry 2011 can be coupled to various sensors including, but not limited to, one or more Light Emitting Diodes (LEDs) or other light emitters, one or more photodiodes or other light sensors, one or more photothermal sensors, a magnetometer, an accelerometer, a gyroscope, a barometer, a compass, a proximity sensor, a camera, an ambient light sensor, a thermometer, a GPS sensor, and various system sensors which can sense remaining battery life, power consumption, processor speed, CPU load, and the like.

Peripherals interface 2016 can couple the input and output peripherals of the system to processor 2018 and computer-readable medium 2001. One or more processors 2018 communicate with one or more computer-readable mediums 2001 via controller 2020. Computer-readable medium 2001 can be any device or medium that can store code and/or data for use by one or more processors 2018. In some examples, medium 2001 can be a non-transitory computer-readable storage medium. Medium 2001 can include a memory hierarchy, including but not limited to cache, main memory and secondary memory. The memory hierarchy can be implemented using any combination of RAM (e.g., SRAM, DRAM, DDRAM), ROM, FLASH, magnetic and/or optical storage devices, such as disk drives, magnetic tape, CDs (compact disks) and DVDs (digital video discs). Medium 2001 may also include a transmission medium for carrying information-bearing signals indicative of computer instructions or data (with or without a carrier wave upon which the signals are modulated). For example, the transmission medium may include a communications network, including but not limited to the Internet (also referred to as the World Wide Web), intranet(s), Local Area Networks (LANs), Wide Local Area Networks (WLANs), Storage Area Networks (SANs), Metropolitan Area Networks (MAN) and the like.

One or more processors 2018 can run various software components stored in medium 2001 to perform various functions for system 2000. In some examples, the software components can include operating system 2022, communication module (or set of instructions) 2024, touch processing module (or set of instructions) 2026, graphics module (or set of instructions) 2028, and one or more applications (or set of instructions) 2030. Each of these modules and above noted applications can correspond to a set of instructions for performing one or more functions described above and the methods described in this application (e.g., the computer-implemented methods and other information processing methods described herein). These modules (i.e., sets of instructions) need not be implemented as separate software programs, procedures or modules, and thus various subsets of these modules may be combined or otherwise re-arranged in various examples. In some examples, medium 2001 may store a subset of the modules and data structures identified above. Furthermore, medium 2001 may store additional modules and data structures not described above.

Operating system 2022 can include various procedures, sets of instructions, software components and/or drivers for controlling and managing general system tasks (e.g., memory management, storage device control, power management, etc.) and facilitates communication between various hardware and software components.

Communication module 2024 can facilitate communication with other devices over one or more external ports 2036 or via RF circuitry 2008 and can include various software components for handling data received from RF circuitry 2008 and/or external port 2036.

Graphics module 2028 can include various known software components for rendering, animating and displaying graphical objects on a display surface. In examples in which touch I/O device 2012 is a touch sensing display (e.g., touch screen), graphics module 2028 can include components for rendering, displaying, and animating objects on the touch sensing display.

One or more applications 2030 can include any applications installed on system 2000, including without limitation, a browser, address book, contact list, email, instant messaging, word processing, keyboard emulation, widgets, JAVA-enabled applications, encryption, digital rights management, voice recognition, voice replication, location determination capability (such as that provided by the global positioning system (GPS)), a music player, etc.

Touch processing module 2026 can include various software components for performing various tasks associated with touch I/O device 2012 including but not limited to receiving and processing touch input received from I/O device 2012 via touch I/O device controller 2032.

I/O subsystem 2006 can be coupled to touch I/O device 2012 and one or more other I/O devices 2014 for controlling or performing various functions. Touch I/O device 2012 can communicate with processing system 2004 via touch I/O device controller 2032, which can include various components for processing user touch input (e.g., scanning hardware). One or more other input controllers 2034 can receive/send electrical signals from/to other I/O devices 2014. Other I/O devices 2014 may include physical buttons, dials, slider switches, sticks, keyboards, touch pads, additional display screens, or any combination thereof.

If embodied as a touch screen, touch I/O device 2012 can display visual output to the user in a GUI. The visual output may include text, graphics, video, and any combination thereof. Some or all of the visual output may correspond to user-interface objects. Touch I/O device 2012 can form a touch sensing surface that accepts touch input from the user. Touch I/O device 2012 and touch screen controller 2032 (along with any associated modules and/or sets of instructions in medium 2001) can detect and track touches or near touches (and any movement or release of the touch) on touch I/O device 2012 and can convert the detected touch input into interaction with graphical objects, such as one or more user-interface objects. In the case in which device 2012 is embodied as a touch screen, the user can directly interact with graphical objects that are displayed on the touch screen. Alternatively, in the case in which device 2012 is embodied as a touch device other than a touch screen (e.g., a touch pad), the user may indirectly interact with graphical objects that are displayed on a separate display screen embodied as I/O device 2014.

Touch I/O device 2012 may be analogous to the multi-touch sensing surface described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference.

In examples for which touch I/O device 2012 is a touch screen, the touch screen may use LCD (liquid crystal display) technology, LPD (light emitting polymer display) technology, OLED (organic LED), or OEL (organic electro luminescence), although other display technologies may be used in other examples.

Feedback may be provided by touch I/O device 2012 based on the user's touch input as well as a state or states of what is being displayed and/or of the computing system. Feedback may be transmitted optically (e.g., light signal or displayed image), mechanically (e.g., haptic feedback, touch feedback, force feedback, or the like), electrically (e.g., electrical stimulation), olfactory, acoustically (e.g., beep or the like), or the like or any combination thereof and in a variable or non-variable manner.

System 2000 can also include power system 2044 for powering the various hardware components and may include a power management system, one or more power sources, a recharging system, a power failure detection circuit, a power converter or inverter, a power status indicator and any other components typically associated with the generation, management and distribution of power in portable devices.

In some examples, peripherals interface 2016, one or more processors 2018, and memory controller 2020 may be implemented on a single chip, such as processing system 2004. In some other examples, they may be implemented on separate chips.

Examples of the disclosure can be advantageous in allowing for an electronic device to obtain a heart rate signal with reduced noise due to motion artifacts, making for a more accurate reading of heart rate.

In some examples, a method of an electronic device including a plurality of light emitters and a light sensor may be disclosed. The method may include: emitting light from a first light emitter; receiving at the light sensor first light information from the first light emitter; emitting light from a second light emitter; receiving at the light sensor second light information from the second light emitter; computing first and second scaling factors based on the first and second light information; and computing a heart rate signal based on the first light information added to the second light information, the first and second light information each being scaled by the respective first and second scaling factors. Additionally or alternatively to one or more of the examples discussed above, the first light emitter may be located a first distance from the light sensor along a surface of the electronic device, and the second light emitter may be located a second distance from the light sensor along a surface of the electronic device; wherein the second distance may be greater than the first distance. Additionally or alternatively to one or more of the examples discussed above, an emission pattern of the first light emitter may have a first angle of emission and an emission pattern of the second light emitter may have a second angle of emission; wherein the second angle of emission may be more acute than the first angle of emission. Additionally or alternatively to one or more of the examples discussed above, the computing of the first and second scaling factors may include optimization based on a previously computed heart rate signal. Additionally or alternatively to one or more of the examples discussed above, computing the heart rate signal may include cancelling noise due to changes in a gap between the electronic device and tissue of a user. Additionally or alternatively to one or more of the examples discussed above, the first light emitter may emit light having a first wavelength and the second light emitter may emit light having a second wavelength, the first wavelength being different from the second wavelength. Additionally or alternatively to one or more of the examples discussed above, the first light emitter may emit light during a first period and the second light emitter may emit light during a second period different from the first period, and wherein the first light information may be received during the first period and the second light information may be received during the second period.

In some examples, a method of reducing noise in a reflected light signal may be disclosed. The method may include: receiving a plurality of reflected light signals generated from a plurality of light emitters and reflected by a first surface; and computing the reflected light signal from the plurality of reflected light signals while canceling noise in the computed reflected light signal due to estimated changes in a gap between the plurality of light emitters and the first surface based on the plurality of reflected light signals.

In some examples, a non-transitory computer readable medium may be disclosed, the computer readable medium containing instructions that, when executed, perform a method of an electronic device including a plurality of light emitters and a light sensor. The method may include: emitting light from a first light emitter; receiving at the light sensor first light information from the first light emitter; emitting light from a second light emitter; receiving at the light sensor second light information from the second light emitter; computing first and second scaling factors based on the first and second light information; and computing a heart rate signal based on the first light information added to the second light information, the first and second light information each being scaled by the respective first and second scaling factors. Additionally or alternatively to one or more of the examples discussed above, the first light emitter may be located a first distance from the light sensor along a surface of the electronic device, and the second light emitter may be located a second distance from the light sensor along a surface of the electronic device; wherein the second distance may be greater than the first distance. Additionally or alternatively to one or more of the examples discussed above, an emission pattern of the first light emitter may have a first angle of emission and an emission pattern of the second light emitter may have a second angle of emission; wherein the second angle of emission may be more acute than the first angle of emission. Additionally or alternatively to one or more of the examples discussed above, the computing of the first and second scaling factors may include optimization based on a previously computed heart rate signal. Additionally or alternatively to one or more of the examples discussed above, computing the heart rate signal may include cancelling noise due to changes in a gap between the electronic device and tissue of a user. Additionally or alternatively to one or more of the examples discussed above, the first light emitter may emit light having a first wavelength and the second light emitter may emit light having a second wavelength, the first wavelength being different from the second wavelength. Additionally or alternatively to one or more of the examples discussed above, the first light emitter may emit light during a first period and the second light emitter may emit light during a second period different from the first period, and wherein the first light information may be received during the first period and the second light information may be received during the second period.

In some examples, a non-transitory computer readable medium may be disclosed, the computer readable medium containing instructions that, when executed, perform a method of reducing noise in a reflected light signal. The method may include: receiving a plurality of reflected light signals generated from a plurality of light emitters and reflected by a first surface; and computing the reflected light signal from the plurality of reflected light signals while canceling noise in the computed reflected light signal due to estimated changes in a gap between the plurality of light emitters and the first surface based on the plurality of reflected light signals.

In some examples, an electronic device may be disclosed. The electronic device may include: a processor to execute instructions; a plurality of light emitters; a light sensor; and a memory coupled with the processor to store instructions, which when executed by the processor, cause the processor to perform operations to generate an application programming interface (API) that allows an API-calling component to perform a method. The method may include: emitting light from a first light emitter; receiving at the light sensor first light information from the first light emitter; emitting light from a second light emitter; receiving at the light sensor second light information from the second light emitter; computing first and second scaling factors based on the first and second light information; and computing a heart rate signal based on the first light information added to the second light information, the first and second light information each being scaled by the respective first and second scaling factors. Additionally or alternatively to one or more of the examples discussed above, the first light emitter may be located a first distance from the light sensor along a surface of the electronic device, and the second light emitter may be located a second distance from the light sensor along a surface of the electronic device; wherein the second distance may be greater than the first distance. Additionally or alternatively to one or more of the examples discussed above, an emission pattern of the first light emitter may have a first angle of emission and an emission pattern of the second light emitter may have a second angle of emission; wherein the second angle of emission may be more acute than the first angle of emission. Additionally or alternatively to one or more of the examples discussed above, the computing of the first and second scaling factors may include optimization based on a previously computed heart rate signal. Additionally or alternatively to one or more of the examples discussed above, computing the heart rate signal may include cancelling noise due to changes in a gap between the electronic device and tissue of a user. Additionally or alternatively to one or more of the examples discussed above, the first light emitter may emit light having a first wavelength and the second light emitter may emit light having a second wavelength, the first wavelength being different from the second wavelength. Additionally or alternatively to one or more of the examples discussed above, the first light emitter may emit light during a first period and the second light emitter may emit light during a second period different from the first period, and wherein the first light information may be received during the first period and the second light information may be received during the second period.

In some examples, an electronic device may be disclosed. The electronic device may include: a processor to execute instructions; a plurality of light emitters; and a memory coupled with the processor to store instructions, which when executed by the processor, cause the processor to perform operations to generate an application programming interface (API) that allows an API-calling component to perform a method of reducing noise in a reflected light signal. The method may include: receiving a plurality of reflected light signals generated from a plurality of light emitters and reflected by a first surface; and computing the reflected light signal from the plurality of reflected light signals while canceling noise in the computed reflected light signal due to estimated changes in a gap between the plurality of light emitters and the first surface based on the plurality of reflected light signals.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

1. A method of an electronic device including a plurality of light emitters and a light sensor, the method comprising:
emitting light from a first light emitter;
receiving at the light sensor first light information from the first light emitter;
emitting light from a second light emitter;
receiving at the light sensor second light information from the second light emitter;
computing first and second scaling factors based on the first and second light information; and
computing a heart rate signal based on the first light information added to the second light information, the first and second light information each being scaled by the respective first and second scaling factors.

2. The method of claim 1, wherein the first light emitter is located a first distance from the light sensor along a surface of the electronic device, and the second light emitter is located a second distance from the light sensor along a surface of the electronic device;
wherein the second distance is greater than the first distance.

3. The method of claim 1, wherein an emission pattern of the first light emitter has a first angle of emission and an emission pattern of the second light emitter has a second angle of emission;
wherein the second angle of emission is more acute than the first angle of emission.

4. The method of claim 1, wherein the computing of the first and second scaling factors includes optimization based on a previously computed heart rate signal.

5. The method of claim 1, wherein computing the heart rate signal includes cancelling noise due to changes in a gap between the electronic device and tissue of a user.

6. The method of claim 1, wherein the first light emitter emits light having a first wavelength and the second light emitter emits light having a second wavelength, the first wavelength being different from the second wavelength.

7. The method of claim 1, wherein the first light emitter emits light during a first period and the second light emitter emits light during a second period different from the first period, and wherein the first light information is received during the first period and the second light information is received during the second period.

8.-14. (canceled)

15. An electronic device, comprising:
a first light emitter configured to emit light;
a first light sensor configured to receive first light information from the first light emitter;
a second light emitter configured to emit light, wherein the first light sensor is further configured to receive second light information from the second light emitter; and
a processor configured to:
compute first and second scaling factors based on the first and second light information; and
compute a heart rate signal based on the first light information added to the second light information, the first and second light information each being scaled by the respective first and second scaling factors.

16. The electronic device of claim 15, wherein the first light emitter is located a first distance from the first light sensor along a surface of the electronic device, and the second light emitter is located a second distance from the first light sensor along the surface of the electronic device;
wherein the second distance is greater than the first distance.

17. The electronic device of claim 15, wherein an emission pattern of the first light emitter has a first angle of emission and an emission pattern of the second light emitter has a second angle of emission;
wherein the second angle of emission is more acute than the first angle of emission.

18. The electronic device of claim 15, wherein the computation of the first and second scaling factors includes optimization based on a previously computed heart rate signal.

19. The electronic device of claim 15, wherein the computation of the heart rate signal includes cancelling noise due to changes in a gap between the electronic device and a tissue of a user.

20. The electronic device of claim 15, wherein the first light emitter emits light having a first wavelength and the second light emitter emits light having a second wavelength, the first wavelength being different from the second wavelength.

21. The electronic device of claim 15, wherein the first light emitter emits light during a first period and the second light emitter emits light during a second period different from the first period, and wherein the first light information is received during the first period and the second light information is received during the second period.

22. A method of reducing noise in a reflected light signal, the method comprising:
receiving a plurality of reflected light signals generated from a plurality of light emitters and reflected by a first surface; and
computing the reflected light signal from the plurality of reflected light signals while canceling noise in the computed reflected light signal due to estimated changes in a gap between the plurality of light emitters and the first surface based on the plurality of reflected light signals.

23.-24. (canceled)

25. The electronic device of claim 15, further comprising:
a second light sensor configured to receive third light information from the first light emitter and configured to receive fourth light information from the second light emitter,
wherein the processor is further configured to:
compute third and fourth scaling factors based on the third and fourth light information, and
wherein the heart rate signal is further computed based on the third and fourth light information each being scaled by the respective third and fourth scaling factors.

26. The electronic device of claim 25, further comprising:
a third light emitter configured to emit light;
a third light sensor configured to receive fifth light information from the third light emitter; and
a fourth light emitter configured to emit light, wherein the third light sensor is further configured to receive sixth light information from the fourth light emitter,
wherein the processor is further configured to:
compute fifth and sixth scaling factors based on the fifth and sixth light information, and
wherein the heart rate signal is further computed based on the fifth and sixth light information being scaled by the respective fifth and sixth scaling factors.

27. The electronic device of claim 26, further comprising:
a fourth light sensor configured to receive seventh light information from the third light emitter and configured to receive eighth light information from the fourth light emitter,
wherein the processor is further configured to:
compute seventh and eighth scaling factors based on the seventh and eighth light information, and
wherein the heart rate signal is further computed based on the seventh and eighth light information each being scaled by the respective seventh and eighth scaling factors.

28. The electronic device of claim 27, wherein an optical axis of the first light emitter, second light emitter, first light sensor, and second light sensor intersects with an optical axis of the third light emitter, fourth light emitter, third light sensor, and fourth light sensor.

29. The electronic device of claim 15, wherein the computation of the first and second scaling factors includes optimization based on previously computed first and second light information.
